(19) United States
(12) Patent Application Publication
(10) Pub. No.: US 2025/0248711 A1
(43) Pub. Date: Aug. 7, 2025

(54) REPOSITIONABLE OVER-THE-SCOPE CLIP

(71) Applicant: BOSTON SCIENTIFIC MEDICAL DEVICE LIMITED, Galway (IE)

(72) Inventor: Deepak Kumar SHARMA, Muzaffarnagar (IN)

(21) Appl. No.: 19/025,100

(22) Filed: Jan. 16, 2025

Related U.S. Application Data
(60) Provisional application No. 63/550,359, filed on Feb. 6, 2024.

(51) Int. Cl. A61B 17/128; A61B 1/00; A61B 17/00; A61B 17/122; A61B 17/30

(52) U.S. Cl. CPC A61B 17/1285; A61B 1/00101; A61B 17/122; A61B 2017/00296; A61B 2017/00367; A61B 2017/00902; A61B 2017/306

(57) ABSTRACT

A clipping device includes a cap having inner and outer bodies separated by a gap, a clip, a control wire extending from proximal to distal ends, and first and second sheaths. The clip is mounted on a structure and biased toward a closed configuration. The clip is received on the inner body with the arms separated to draw tissue. The first sheath receives the wire and extends to a distal end movable between insertion and extended positions. The second sheath slidably receives the first sheath and extends from a proximal end to a distal end fixed relative to the inner body. The structure extends beyond a distal end of the inner body to engage a central portion of the clip as the clip is drawn toward the cap so that the structure forces the arms apart to draw the clip into the gap.

### PRIORITY CLAIM

The present disclosure claims priority to U.S. Provisional Patent Application Ser. No. 63/550,359 filed Feb. 6, 2024; the disclosure of which is incorporated herewith by reference

### FIELD

The present disclosure relates to endoscopic devices and, in particular, relates to an endoscopic clipping device for treating tissue along the gastrointestinal tract.

### BACKGROUND

Some endoscopic procedures (e.g., the removal of larger lesions (e.g., cancerous and other)); tunneling under the mucosal layer of the GI tract to treat issues below the mucosa; full thickness removal of tissue (e.g., cancerous and other); treatment of issues on other organs by passing outside of the GI Tract (Natural Orifice Transluminal Endoscopic Surgery aka NOTES®); and endoscopic treatment/repair of post-surgical issues (e.g., post-surgical leaks, breakdown of surgical staple lines and/or anastomotic leaks); may increase the risk, in certain cases, of, for example, intrabdominal hypertension and seeding of cancer cells. In addition, these procedures generally create tissue openings that need to be hemo-statically sealed.

### SUMMARY

The present disclosure relates to a clipping device for treating tissue within a living body. The device includes a cap configured to be coupled to a distal end of an insertion instrument, the cap including an inner body defining an inner channel and an outer body surrounding the inner body and separated from the inner body by a gap. Also, the device includes a first clip configured to be slidably mounted on a first mounting structure. The first clip is biased toward a closed configuration in which arms of the first clip are drawn toward one another to clip tissue received therebetween, the first clip being, in an insertion configuration, slidably received on the inner body with the arms of the first clip being held separated from one another by the inner body so that tissue drawn into the channel of the inner body is drawn between the arms of the first clip.

In addition, the device includes a first control wire extending from a proximal end configured to remain outside the living body while the insertion instrument and the cap are inserted into the living body, the first control wire extending to a distal end separably coupled to the first clip. Furthermore, the device includes a first sheath slidably receiving the first control wire therein and extending to a distal end movable between an insertion position in which the distal end of the first sheath is located on the inner body and an extended position in which the distal end of the first sheath extends distally beyond the distal end of the inner body.

In addition, the device includes a second sheath slidably receiving the first sheath and extending from a proximal end configured to remain outside the living body to a distal end fixed relative to the inner body, the first mounting structure extending distally beyond a distal end of the inner body to engage a central portion of the first clip as the first clip is drawn proximally toward the cap so that the first mounting structure forces the arms of the first clip apart from one another into a reload configuration in which the first clip may be drawn into the gap between the inner and outer bodies.

In an embodiment, a distal end of the outer body is configured to engage the arms of the first clip in the reload configuration to force the arms of the first clip back into the insertion configuration as the first clip is drawn proximally into the gap.

In an embodiment, the distal end of the outer body flares outward compared to a proximal part of the outer body.

In an embodiment, the arms of the first clip are substantially linear in the reload configuration and wherein the first mounting structure is configured to engage the central portion of the first clip as the first clip is drawn proximally over the first mounting structure so that the arms of the first clip are separated from one another into a V shape in the reload configuration.

In an embodiment, the channel of the inner body is configured so that, when the cap is mounted on an insertion device as desired, the distal end of the insertion device is open to the channel and wherein the channel extends to an open distal end so that tissue drawn into the channel is drawn between the arms of the first clip when the first clip is mounted on the inner body in the insertion configuration.

In an embodiment, the inner and outer bodies are formed of a transparent material.

In an embodiment, the central portion of the first clip is coupled to a first one of the arms of the first clip via a first connection part and the central portion of the first clip is coupled to a second one of the arms of the first clip via a second connection part, the central portion of the first clip being configured to bias the first clip toward the closed configuration and the first and second connection parts of the first clip being configured to engage laterally outer portions of the first mounting structure to drive the first clip to the reload configuration.

In an embodiment, the first clip includes a wire mounting structure having an opening through which the first control wire passes to separably couple the first clip to the first control wire, the first control wire including a flange at a distal end thereof that is larger than the opening in the wire mounting structure of the first clip.

In an embodiment, the wire mounting structure is designed to fail when subject to a proximally directed force of at least a predetermined level by the flange of the first control wire.

In an embodiment, the predetermined level of the proximally directed force is less than an amount of a proximally directed force required to draw the first clip proximally onto the inner body.

In an embodiment, the first sheath is extendable into contact with a proximal surface of the wire mounting structure of the first clip to prevent proximal movement of the first clip so that a proximally directed force applied to the first control wire can be raised above the predetermined level.

In an embodiment, the inner body further includes a second mounting structure. The device further includes a second clip configured to be slidably mounted on the second mounting structure, the second clip being biased toward the closed configuration in which arms of the second clip are drawn toward one another to clip tissue received therebetween, the second clip being, in an insertion configuration, slidably received on the inner body with the arms of the second clip being held separated from one another by the inner body so that tissue drawn into the channel of the inner body is drawn between the arms of the second clip; a second control wire extending from a proximal end configured to remain outside the living body while the insertion instrument and the cap are inserted into the living body, the second control wire extending to a distal end separably coupled to the second clip; a third sheath slidably receiving the second control wire therein and extending to a distal end movable between an insertion position in which the distal end of the third sheath is located on the inner body and an extended position in which the distal end of the third sheath extends distally beyond the distal end of the inner body;

and a fourth sheath slidably receiving the third sheath and extending from a proximal end configured to remain outside the living body to a distal end fixed relative to the inner body, the second mounting structure extending distally beyond a distal end of the inner body to engage the central portion of the second clip as the second clip is drawn proximally toward the cap so that the second mounting structure forces the arms of the second clip apart from one another into a reload configuration in which the second clip may be drawn into the gap between the inner and outer bodies.

In an embodiment, the first and second mounting structures are located on the inner body diametrically opposed to one another.

In addition, the present disclosure relates to a clipping device for treating tissue within a living body. The device includes a cap configured to be coupled to a distal end of an insertion instrument, the cap including an inner body defining an inner channel and an outer body surrounding the inner body and separated from the inner body by a gap, the inner body further including a first mounting structure extending over a first portion of a circumference of the inner body and projecting distally beyond a distal end of a second portion of the circumference of the inner body; a first clip configured to be slidably mounted on the first mounting structure, the first clip being biased toward a closed configuration in which arms of the first clip are drawn toward one another to clip tissue received therebetween, the first clip being, in an insertion configuration, slidably received on the inner body with the arms of the first clip being held separated from one another by the inner body so that tissue drawn into the channel of the inner body is drawn between the arms of the first clip, the first mounting structure being configured to engage a central portion of the first clip as the first clip is drawn proximally toward the cap so that the first mounting structure forces the arms of the first clip apart from one another into a reload configuration in which the first clip may be drawn into the gap between the inner and outer bodies; and a first control wire extending to a distal end separably coupled to the first clip, the first control wire being operable to move the first clip proximally and distally relative to the inner body to move the clip between the insertion configuration, the closed configuration and the reload configuration.

In an embodiment, a distal end of the outer body is configured to engage the arms of the first clip in the reload configuration to force the arms of the first clip back into the insertion configuration as the first clip is drawn proximally into the gap.

In addition, the present disclosure relates to a method for clipping tissue. The method includes inserting to a target location within a living body a clipping device, comprising a cap coupled to a distal end of a flexible insertion instrument with a first clip stretched over an inner body of the cap in an insertion configuration, wherein the inner body further includes a first mounting structure extending over a first portion of a circumference of the inner body and projecting distally beyond a distal end of a second portion of the circumference of the inner body; drawing a portion of tissue into a channel defined by the inner body so that a first portion of tissue is drawn between arms of the first clip; pushing the first clip distally so that the arms of the first clip are drawn together under a natural bias of the first clip to clip the portion of tissue drawn into the channel; observing a position of the first clip on the portion of tissue to determine if a first target tissue has been clipped as desired and, when the first target tissue has not been clipped as desired, drawing the first clip proximally so that engagement between a central portion of the first clip and the first mounting structure forces the arms of the first clip apart from one another into a reload configuration to release the portion of tissue; drawing the first clip further proximally into a gap extending between the inner body and an outer body of the cap, engagement between the arms of the first clip and the outer body forcing the first clip back into the insertion configuration; repositioning the cap relative to the first target tissue and drawing into the channel a further portion of tissue; and pushing the first clip distally so that the arms of the first clip are drawn together to clip the further portion of tissue.

In an embodiment, the clipping device includes a first control wire connected to the first clip for moving the first clip between the insertion configuration, a closed configuration and the reload configuration further comprising, when it is desired to fully deploy the first clip, applying to a connection between the first control wire and the first clip a tension greater than a first predetermined tension to permanently separate the first clip from the first control wire.

In an embodiment, the first predetermined tension is greater than a tension applied to the connection between the first control wire and the first clip when the first clip is drawn proximally from the closed configuration to the reload configuration and a tension applied to the connection between the first control wire and the first clip when the first clip is drawn proximally from the reload configuration to the insertion configuration.

In an embodiment, the wherein the inner body further includes a second mounting structure and wherein the clipping device further includes a second clip mounted on the second mounting structure in an insertion configuration with the arms of the second clip held separated from one another by the inner body, the method further comprising: drawing into the channel a still further portion of tissue; operating a second control wire coupled to the second clip to push the second clip distally off of the inner body so that the arms of the second clip close over the still further portion of tissue; observing a position of the second clip on the still further portion of tissue to determine if a second target tissue has been clipped as desired and, when the second target tissue has not been clipped as desired, operating the second control wire to draw the second clip proximally so that engagement between a central portion of the second clip and the second mounting structure forces the arms of the second clip apart from one another into a reload configuration to release the still further portion of tissue; operating the second control wire to draw the second clip further proximally into the gap between the inner body and the outer body of the cap, engagement between the arms of the second clip and the outer body forcing the second clip back into the insertion configuration; repositioning the cap relative to the second target tissue and drawing into the channel an additional portion of tissue; and operating the second control wire to push the second clip distally so that the arms of the second clip are drawn together to clip the additional portion of tissue.

In an embodiment, the method further includes operating the second control wire to apply to a connection between the second control wire and the second clip a tension greater than a predetermined tension to separate the second clip from the second control wire.

### BRIEF DESCRIPTION OF THE DRAWINGS

FIG. 1 shows a perspective view of a distal portion of a system according to an exemplary embodiment in an insertion configuration;

FIG. 2 shows a perspective view of a cap of the system of FIG. 1;

FIG. 3 shows a perspective view of the distal portion of the system of FIG. 1 with the clip moved off of the cap to a closed configuration;

FIG. 4 shows a front view of a clip of the system of FIG. 1 in the closed configuration;

FIG. 5 shows a perspective view of a handle of the system of FIG. 1;

FIG. 6 shows a front view of a clip of the system of FIG. 1 in a closed configuration;

FIG. 7 shows a perspective view of the clip of FIG. 6 attached to a control wire of the system of FIG. 1;

FIG. 8 shows a perspective view of the distal portion of the system of FIG. 1 in clip a tissue receiving configuration;

FIG. 9 shows a perspective view of the distal portion of the system of FIG. 1 in a review configuration;

FIG. 10 shows a perspective view of the distal portion of the system of FIG. 1 with the clip

finally deployed;

FIG. 11 shows a perspective view of the distal portion of a system according to a further embodiment including two clips loaded in the cap;

FIG. 12 shows a perspective view of the distal portion of the system of FIG. 11 showing a conical flare at a distal end of an outer body of a cap with no clips loaded therein;

FIG. 13 shows a perspective view of the distal portion of the system of FIG. 11 showing a first clip extended out of the cap;

FIG. 14 shows a perspective view of the distal portion of the system of FIG. 11 with a clip in the reload configuration;

FIG. 15 shows a perspective view of the distal portion of the system of FIG. 11 with a clip withdrawn over the inner body in the insertion configuration;

FIG. 16 shows a perspective view of the distal portion of the system of FIG. 11 with a clip

fully deployed therefrom;

FIG. 17 shows a perspective view of the distal portion of the system of FIG. 11 with a first clip deployed therefrom and a second clip extended to the reload configuration;

FIG. 18 shows a perspective view of the distal portion of the system of FIG. 11 with the second clip extended to the review configuration; and

FIG. 19 shows a perspective view of the distal portion of the system of FIG. 11 with the first and second clips fully deployed therefrom.

### DETAILED DESCRIPTION

The present disclosure may be further understood with reference to the following description and the appended drawings, wherein like elements are referred to with the same reference numerals. The present disclosure relates to a clipping system and, in particular, relates to an over-the-scope endoscopic clipping system which may be used, for example, to close tissue openings created through a full thickness resection procedure within the GI tract. In addition, as will be described below, the large clipping size and low profile of the clip makes this clip suitable for closing tissue openings that require more than one clip. That is, the low profile of the clips according to the disclosed embodiments allows more than one of these clips to be placed on tissue of a single wound to be closed.

Exemplary embodiments of the present disclosure comprise a clip that is configured to be mounted on a cap fitted to a distal end of an endoscope with arms of the clip held by the cap in a rounded C-shape. The clips according to these embodiments are biased to assume a closed tissue clipping configuration in which the arms extend generally parallel to one another to clip tissue received between the arms. It should be noted that in this application the terms proximal and distal connote directions along the device toward (proximal) and away (distal) from a user.

A clip according to the disclosed embodiments includes two arms joined to one another by a connecting portion that biases the clip arms toward the closed tissue clipping configuration and which is configured to force the clip arms away from the closed tissue clipping configuration back to the open tissue receiving configuration when pulled by a user proximally back into contact with the cap as will be described in more detail below.

The system includes a cap configured to be mounted over the distal end of an insertion device (e.g., a flexible endoscope) and a control wire configured to permit a user to move the clip proximally and distally relative to a distal end of the cap to move the clip between the fully open configuration when the clip is received on the cap and a closed configuration when the clip has been moved distally off of the distal end of the cap. The control wire of the disclosed embodiments also permits the user to separate the clip from the rest of the system when the user determines that the clip has been clipped to target tissue as desired.

The system also permits the user to move the insertion device away from a clip that has been clipped over a portion of tissue into a review configuration while maintaining a connection between the clip and the rest of the system. In the review configuration, the endoscope is drawn away from the clip to widen the field of view of the endoscopic vision system so that the user may review the position of the clip relative to target tissue (e.g., a tissue to be clipped). If, in the review configuration, it is determined that the clip is not clipped over the target tissue as desired, the clip may be re-opened so that the previously clipped tissue may be released and the cap and the clip may be repositioned relative to the target tissue to restart the clipping process. When the clip is again clipped over tissue and it is observed (e.g., in the review configuration) that the clip is clipped over the target tissue as desired, the clip is finally deployed by separating the clip from the control member.

It will be understood by those of skill in the art that exemplary embodiments of the present disclosure describe a clipping system which may be used to optimize a variety of tissue treatment/resection procedures within the body such as those, for example, performed using a flexible endoscope.

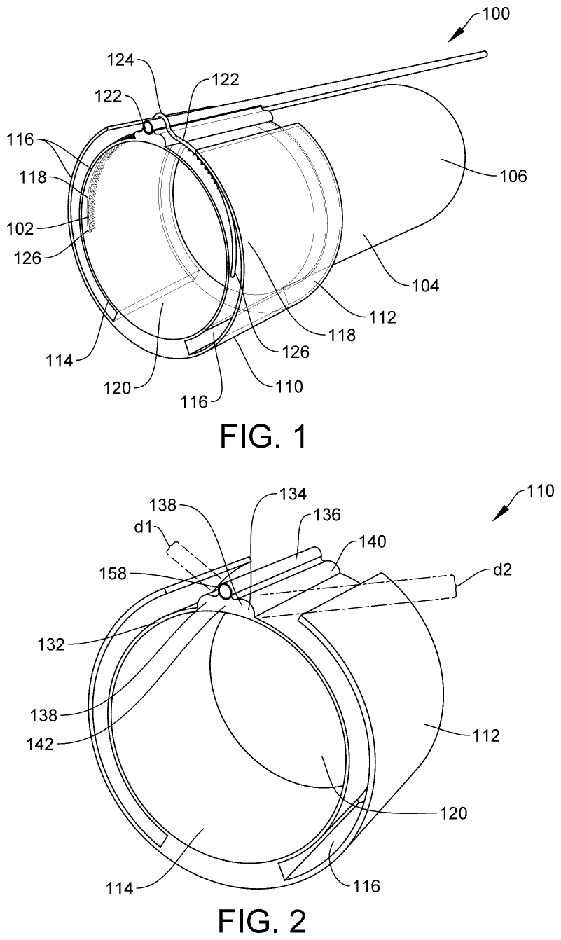

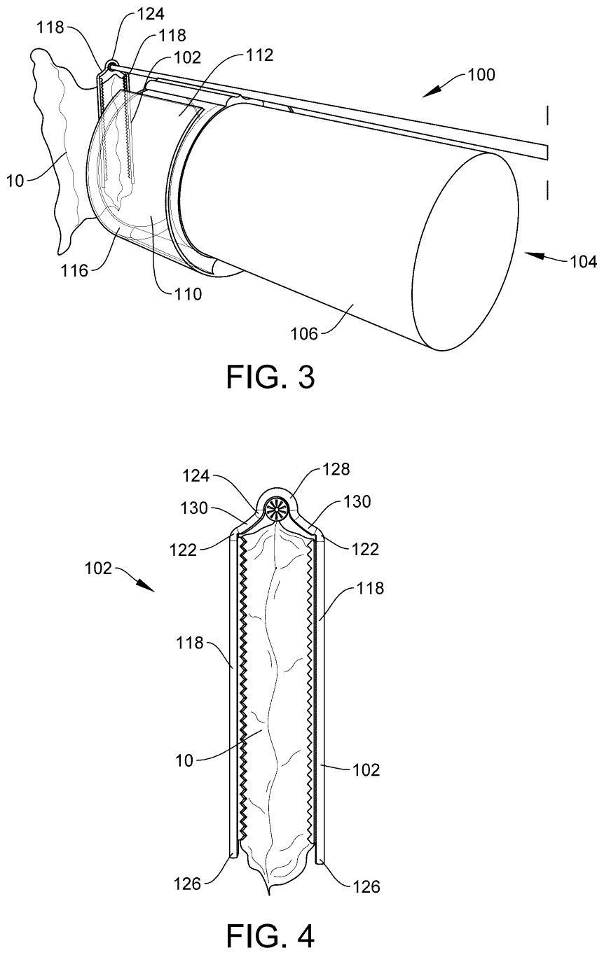

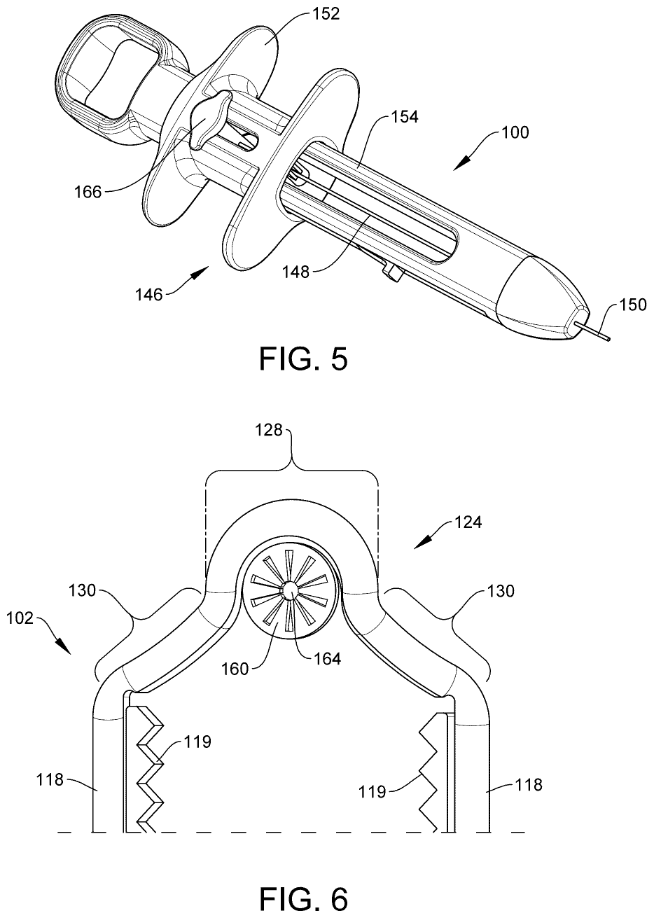

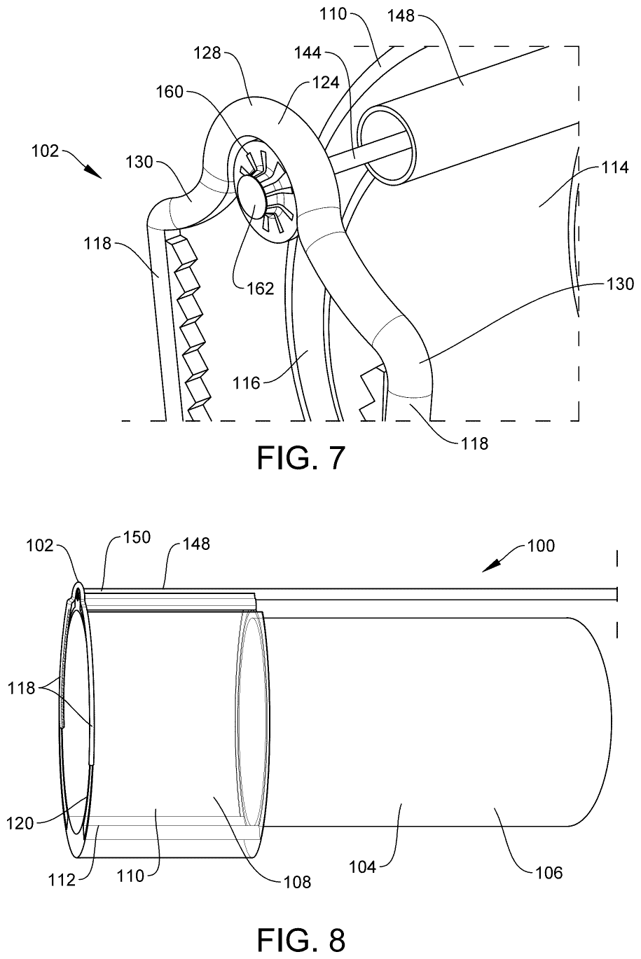

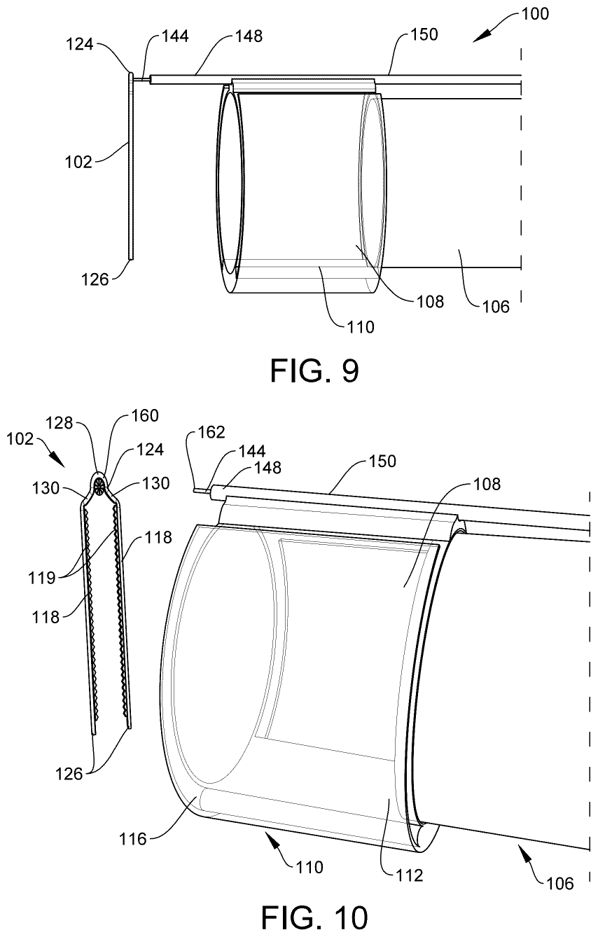

As shown in FIGS. 1-10, a clipping system 100 according to an exemplary embodiment of the present disclosure, comprises a clip 102 configured to be inserted through, for example, a body lumen to a target area to clip target tissue 10 (shown in FIG. 3)—e.g., a target portion of lesion to be clipped, edges of a wound or incision to be healed, etc. The clip 102 is insertable to the target area via an insertion device 104 including, for example, an endoscope 106 and a cap 110 mounted (e.g., via a friction fit) over a distal end 108 of the endoscope 106, as shown in FIG. 1. The cap 110 includes an outer body 112 and an inner body 114 mounted radially within the outer body 112 so that an annular gap 116 is formed between the inner body 114 and the outer body 112.

The clip 102 is configured to be positioned, in an insertion configuration, within the gap 116 so that the inner body 114 holds the clip arms 118 of the clip 102 separated from one another in an open tissue receiving configuration. The inner body 114 defines a channel 120 with an open distal end via which tissue to be clipped may be drawn into the channel 120. The proximal end of the channel 120 is also open so that a working channel of the endoscope 106 opens into the channel 120 permitting a user to apply suction via the working channel into the channel 120 to draw tissue therein. As would be understood by those skilled in the art, alternatively a user may draw tissue into the channel 120 via a grasper or other tool inserted through the endoscope 106 into the channel 120.

Those skilled in the art will understand that the cap 110 including the outer body 112 and the inner body 114 are, in one exemplary embodiment, formed of transparent material to permit the user to see as wide a field of view as possible via the vision system of the endoscope (mounted, e.g., on a distal end of an endoscope 106 (e.g., a flexible endoscope) which, when the cap 110 is mounted over the distal end of the endoscope 106, faces into an interior space of the inner body 114). The cap 110 of this embodiment is mounted over the endoscope 106, for example, so that the channel of the cap 110 is substantially longitudinally aligned with a longitudinal axis of the endoscope 106 to permit target tissue to be viewed through the channel of the cap 110 via the viewing system of the endoscope 106.

Those skilled in the art will recognize that these components do not need to be transparent to function as described herein. This is generally preferred as it can enhance the user's ability to visualize the area surrounding the distal end of the endoscope, but is not necessary. According to an exemplary embodiment, the clip 102 includes clip arms 118 (e.g., two clip arms), each of which extends from a first end 122 coupled to a connection part 124 to a free second end 126. The connection part 124 includes a central curved section 128 that is biased to pull the clip arms 118 toward one another so that, when not constrained by the cap 110, the clip arms 118 move to the closed tissue clipping position as shown in FIG. 4.

The connection part 124 also includes a pair of opening sections 130 configured so that, when the clip 102 is drawn proximally into contact with the distal end 132 of the cap 110 as will be described below in more detail, contact between the opening sections 130 and the distal end 132 of the cap 110 forces the clip arms 118 apart into a reload configuration in which the arms are spread apart from one another into a generally V-shaped configuration, as shown in FIG. 4. The clip 102 of this embodiment is formed of Nitinol with a memorized shape. However, those skilled in the art will understand that the clip may be made of any of a number of suitable biocompatible materials such as, for example, stainless steel or plastics such as, for example, PEEK, Polypropylene, PU, PVC or PC. The clip arms 118 may also include on inner surfaces thereof tissue engaging features 119 (e.g., teeth, irregular surfaces, etc.) configured to enhance the grip of the clip 102 on the clipped tissue.

As will be described in more detail below, the distal end 132 of the cap 110 includes features that permit a user to reload the clip 102 onto the cap 110 if, for example, a user determines that a clip 102 has not been placed as desired on the tissue 10 to be clipped. Those skilled in the art will understand that the shapes of any of the described items may be changed in any manner that does not impede the functioning of the system in placing and deploying the clip 102 as described below. For example, a cap 110 need not be circular but may be elliptical or any other shape configured to couple as desired to an insertion device 104 and to minimize trauma to any tissue contacted by the cap 110 as it is advanced to a target site within the body. Similarly, the clip arms 118 of a clip 102 need not be parallel or even linear when in the closed tissue clipping configuration. Rather, the clip arms 118 may have spatial relation to one another (and need not be symmetric or the same size and/or shape) selected to draw tissue together as desired.

In this embodiment, the clip 102 is, in the open tissue receiving configuration, substantially circular as it is held in this shape by contact with the inner body 114 of the cap 110. This permits an interior space of the clip 102 to be made as large as possible without increasing a maximum cross-sectional extent of the system 100 beyond that of the circular distal end of the outer body 112 of the cap 110. Thus, in this embodiment, an outer diameter of the clip 102 is substantially the same as the inner diameter of the outer body 112.

The distal end 132 of the cap 110 includes a mounting structure 134 that projects radially outward from the outer surface of the inner body 114 and which is shaped and sized to correspond to the size and shape of the connection part 124 when the clip 102 is in the open, tissue receiving configuration. Thus, the mounting structure 134 includes a central section 136 having a channel configured to slidably receive the inner coil 148 and a pair of shoulders 138 projecting laterally outward from the central section 136. Those skilled in the art that, throughout this specification, the term coil denotes simply a sheath and that the inner coil is simply a sheath sized and shaped to receive the control wire therein and to be slidably received within the outer coil.

The inner and outer coils may therefore be formed in any manner so long as the materials are flexible enough to pass along the tortuous paths by which a flexible endoscope (or other insertion instrument) is passed to reach target tissue (e.g., through a natural body lumen) and so that the sheaths possess column strength sufficient to allow a user to apply a desired level of compressive force to the clip via tension applied to the control wire as will be described below. The shoulders 138 project radially outward from the outer surface of the inner body 114 of the cap 110 by a first distance di and the central section 136 extends radially outward from the shoulders 138 by a second distance d2. The mounting structure 134, the shoulders 138 and the central section 136 extend from a proximal end 140 to a distal end 142 that extends distally beyond the distal end of the inner and outer bodies 114, 112, respectively, of the cap 110.

In this embodiment, the mounting structure 134 extends to the proximal end of the cap 110. However, those skilled in the art will understand that the mounting structure 134 may be shortened and is required to extend along only that portion of the length of the cap 110 on which the clip 102 will be received during use. That is, the mounting structure 134 does not need to extend proximally beyond a proximal-most position of the clip 102. As will be discussed in more detail below, if after the clip 102 has been advanced off of the cap 110 and clipped over tissue, a user determines that the clip 102 is not positioned as desired, the user can draw the clip 102 proximally until the clip 102 contacts the mounting structure 134. That is, because the mounting structure 134 projects distally beyond the distal ends of the inner and outer bodies 114, 112, respectively, the clip 102 will first contact the mounting structure 134 as it is drawn proximally back toward the cap 110.

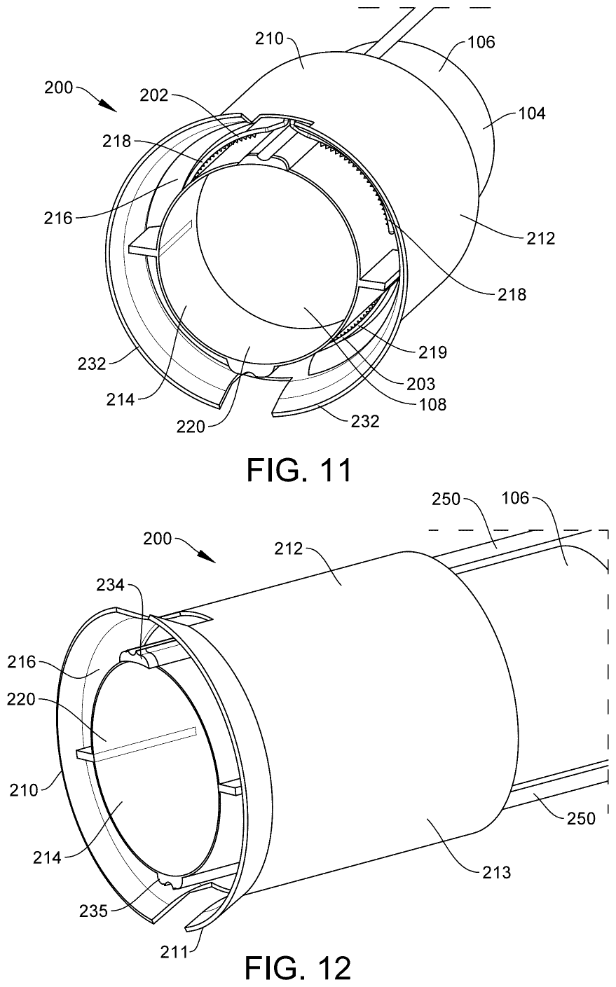

The shape of the mounting structure 134 is selected so that, as the connection part 124 is drawn proximally against the distal end of the mounting structure 134, the clip 102 is forced into an open, reloading configuration with the shoulders 138 forcing the clip arms 118 apart from one another into a substantially V-shaped reloading configuration as shown in FIG. 11. As the connection part 124 is drawn further proximally over the mounting structure 134, the free second ends 126 of the clip arms 118 contact the distal end of the outer body 112 forcing the clip arms 118 to bend so that the free second ends 126 enter the gap 116. As the clip 102 is drawn further proximally, the clip arms 118 are pulled into the gap 116 until the clip 102 returns to the open tissue receiving configuration (i.e., an insertion configuration in which the clip 102 was initially positioned on the cap 110).

The system 100 includes a mechanism for deploying the clip 102 that includes a control wire 144 that extends from a handle 146 that remains outside the patient's body accessible to a user when the distal end of the system 100 is inserted to a target site within the body (e.g., via natural body lumen accessed via a natural body orifice). The control wire 144 extends within an inner coil 148 that is slidably received within an outer coil 150. The inner coil 148 extends from a proximal end coupled to spool 152 that is slidably mounted on a body 154 of the handle 146. The inner coil 148 extends to a distal end 156 that extends through a lumen 158 passing through the mounting structure 134 so that the inner coil 148 is slidably received within the lumen 158.

The outer coil 150 is coupled to the body 154 of the handle 146 extends to a distal end coupled to the proximal end of the mounting structure 134. The outer coil 150 is configured to extend along an outer surface of the insertion device 104 and the control wire 144 extends through the inner coil 148, through the lumen 158 to couple to the clip 102. Specifically, the clip 102 includes a wire mounting structure 160 having an outer diameter substantially equal to an inner diameter of the central curved section 128 and the control wire 144 includes at its distal end an increased diameter flange 162. The control wire 144 extends through the wire mounting structure 160 through a hole 164 therein so that the flange 162 is on the distal side of the wire mounting structure 160. The diameter of the flange 162 is selected to be larger than the diameter of the hole 164 so that movement of the control wire 144 proximally relative to the cap 110 pulls the clip 102 proximally as the flange 162 pushes against the wire mounting structure 160.

The wire mounting structure 160 is configured so that when the control wire 144 imparts to the wire mounting structure 160 a force of no more than a predetermined level, the wire mounting structure 160 prevents the flange 162 from moving proximally therepast. However, the wire mounting structure 160 is constructed so that, when subjected by the flange 162 to a proximally directed force of greater than the predetermined level, the flange 162 will be pulled proximally through the wire mounting structure 160 to completely separate the clip 102 from the rest of the system 100 as will be described in more detail below.

As would be understood by those skilled in the art, the predetermined force is designed to be greater than the level of force that will need to be applied to the clip 102 to draw the clip 102 proximally onto the cap 110 after the clip 102 has been provisionally deployed (e.g., if the user wishes to remove and reposition the clip 102). The control wire 144 extends through the inner coil 148 to couple to a push button 166 that is slidably mounted on the spool 152. As indicated above, the control wire 144 extends through the inner coil 148 from a proximal end coupled to the push button 166 to a distal end coupled to the flange 162. The inner coil 148 passes through the lumen 158 extending through the mounting structure 134 and through the wire mounting structure 160 to the flange 162 which is located on the distal side of the wire mounting structure 160.

The user may then open and close the clip 102 as often as desired by moving the inner coil 148 distally relative to the cap 110 to push the clip 102 distally off of the cap 110 or by moving the inner coil 148 proximally relative to the cap 110 to pull the clip 102 proximally back onto the cap 110 as described above. When the clip 102 has been moved off of the cap 110 and has closed over the tissue 10, the user may then extend the inner coil 148 distally by moving the spool 152 distally relative to the body 154 of the handle 146 while simultaneously drawing the endoscope 106 and the cap 110 proximally. This moves the endoscope 106 proximally away from the clip 102 which remains clipped to the tissue 10 to a review configuration. This, in turn, widens the field of view available to the user who may then move the endoscope 106 as desired to examine the position of the clip 102 to determine if the clip 102 has been deployed as desired.

If the user determines that the clip 102 has not been deployed as desired, the user may then move the endoscope 106 distally while proximally withdrawing the inner coil 148 and the control wire 144 until the clip 102 contacts the mounting structure 134. As described above, further movement of the control wire 144 proximally at this point pulls the clip 102 over the mounting structure 134 to spread the clip arms 118 apart into the reloading configuration. Further proximal movement of the control wire 144 then pulls the clip arms 118 into the gap 116 while the connection part 124 moves proximally over the mounting structure 134 so that the clip returns to the insertion configuration.

As indicated above, the flange 162 at the distal end of the control wire 144 prevents the control wire 144 from being pulled proximally through the wire mounting structure 160. After a user has positioned a clip 102 as desired (e.g., after visually confirming that a target portion of tissue has been clipped by the clip 102 as desired), the user may finally deploy the clip 102 by separating the clip 102 from the system 100. To do this, the user holds the distal end of the inner coil 148 against the proximal surface of the wire mounting structure 160 prevent the clip 102 from moving further proximally.

At the same time, the user applies proximal force to the control wire 144 by sliding the push button 166 proximally relative to the spool 152 until a predetermined tension is applied to the control wire 144 and the flange 162 is pulled through the wire mounting structure 160. When the flange 162 has pulled free from the clip 102, the clip 102 is completely separated from the system 100 and remains closed, clipped over the target tissue under the force of its own natural bias.

A system 200 according to a further embodiment includes a cap configured to be mounted over the distal end of an insertion device (e.g., a flexible endoscope) substantially similar to the system of FIGS. 1-10 except that in this embodiment, the cap includes 2 clips that can be deployed sequentially to work together to close a larger tissue opening or which may be used independently to close 2 tissue openings in substantially the same manner described above. This system 200 includes two control wires each of which is configured to permit a user to move a corresponding one of the clips proximally and distally relative to a distal end of the cap to move the corresponding clip between the fully open configuration when this clip is received on the cap and a closed configuration when this clip has been moved distally off of the distal end of the cap. Each of the control wires of the system 200 also permits the user to separate the corresponding clip from the rest of the system when the user determines that this clip has been clipped over target tissue as desired.

The system 200 also permits the user to move the insertion device (e.g., the endoscope) away from a clip that has been clipped over a portion of tissue into a review configuration while maintaining a connection between the clip and the rest of the system 200. In the review configuration, the endoscope is drawn away from the clip to widen the field of view of a vision system of the endoscope so that the user may review the position of the clip relative to target tissue (e.g., a tissue to be clipped). If, in the review configuration, it is determined that the clip is not clipped over the target tissue as desired, the clip may be re-opened so that the previously clipped tissue may be released and the cap and the clip may be repositioned relative to the target tissue to restart the clipping process. When the clip is again clipped over tissue and it is observed (e.g., in the review configuration) that the clip is clipped over the target tissue as desired, the clip is finally deployed by separating the clip from the corresponding control member.

It will be understood by those of skill in the art that the system 200 may include any of the elements of the system 100 described above and that the system 100 may include any of the elements described in regard to the system 200 excepting those elements which would be redundant or useless for a single clip system.

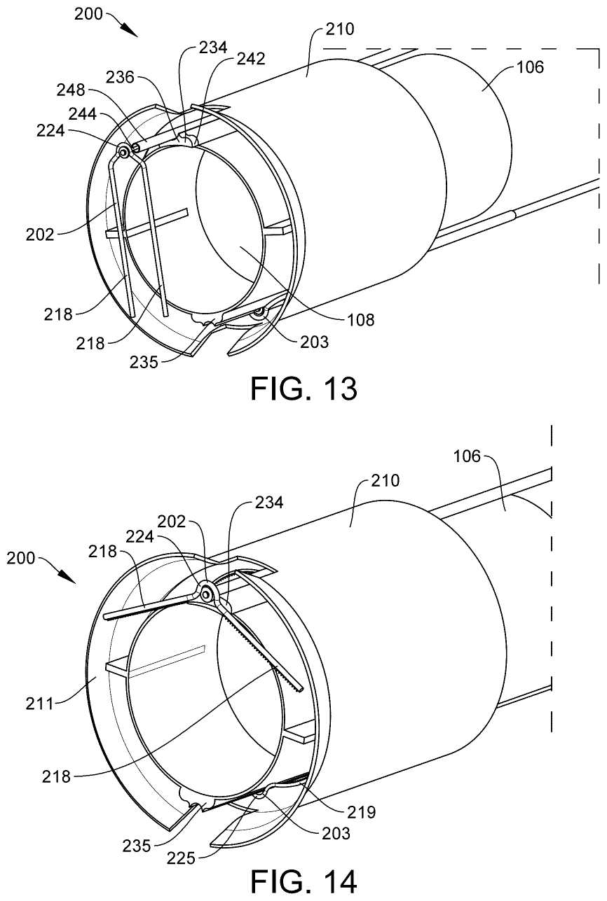

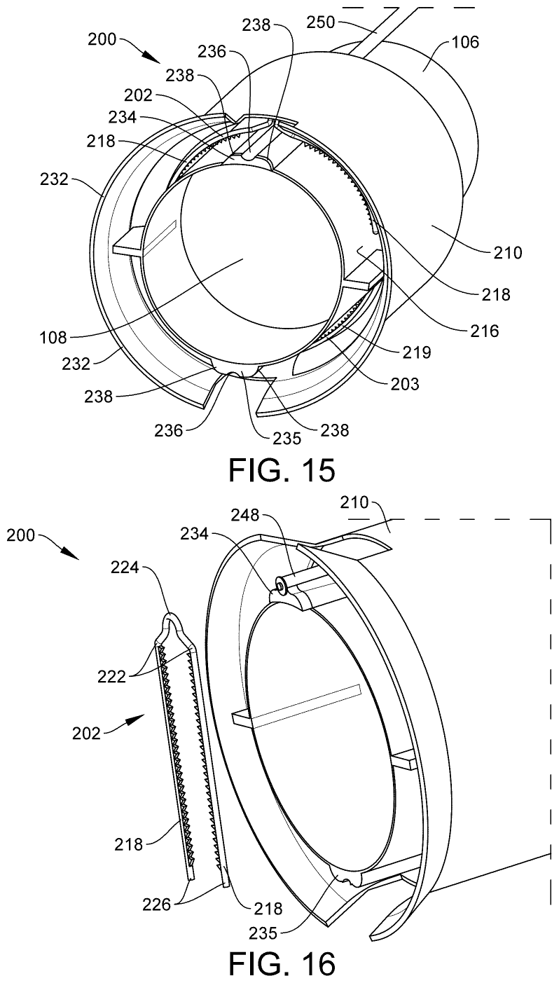

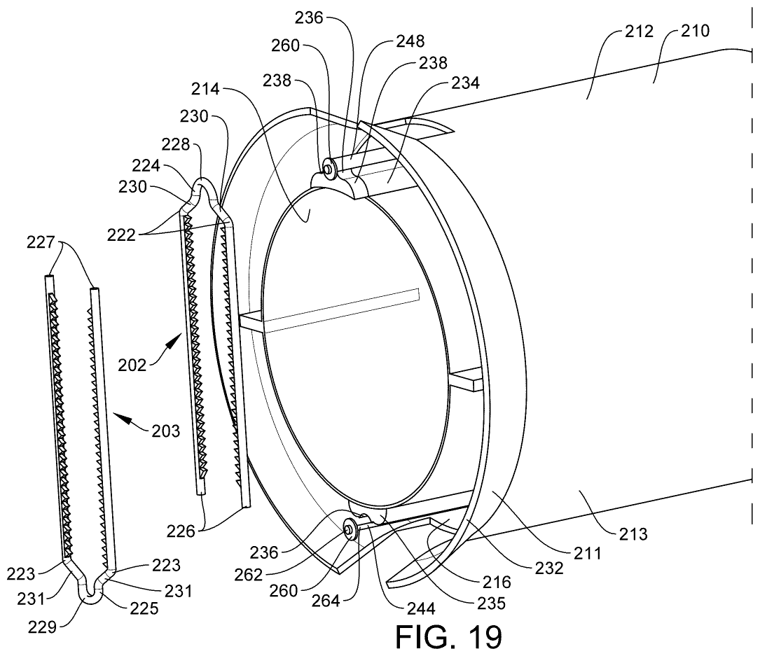

As shown in FIGS. 11-19, a clipping system 200 according to a further exemplary embodiment of the present disclosure, comprises a first clip 202 and a second clip 203 configured to be inserted through, for example, a body lumen to a target area to clip target tissue—e.g., a target portion of lesion to be clipped, edges of a wound or incision to be sealed, etc. The first and second clips 202 and 203 are insertable to the target area via an insertion device 104 including, for example, an endoscope 106 and a cap 210 mounted (e.g., via a friction fit) over a distal end 108 of the endoscope 106, as shown in FIG. 11. The cap 210 includes an outer body 212 and an inner body 214 mounted radially within the outer body 212 so that an annular gap 216 is formed between the inner body 214 and the outer body 212.

The first and second clips 202 and 203 are configured to be positioned, in an insertion configuration, within the gap 216 so that the inner body 214 holds the clip arms 218 of the first clip 202 and the clip arms 219 of the second clip 203 separated from one another in an open tissue receiving configuration. The inner body 214 defines a channel 220 with an open distal end via which tissue to be clipped may be drawn into the channel 220. The proximal end of the channel 220 is also open so that a working channel of the endoscope 106 opens into the channel 220 permitting a user to apply suction via the working channel into the channel 220 to draw tissue therein. As would be understood by those skilled in the art, alternatively a user may draw tissue into the channel 220 via a grasper or other tool inserted through the endoscope 106 into the channel 220.

Those skilled in the art will understand that the cap 210 including the outer body 212 and the inner body 214 may be formed of transparent material to permit the user to see as wide a field of view as possible via the vision system of the endoscope 106 (mounted, e.g., on a distal end of the endoscope 106 which, when the cap 210 is mounted over the distal end of the endoscope 106, faces into an interior space of the inner body 214). The cap 210 of this embodiment is mounted over the endoscope 106, for example, so that the channel 220 of the cap 210 is substantially longitudinally aligned with a longitudinal axis of the endoscope 106 to permit target tissue to be viewed through the channel 220 of the cap 210 via the viewing system of the endoscope 106.

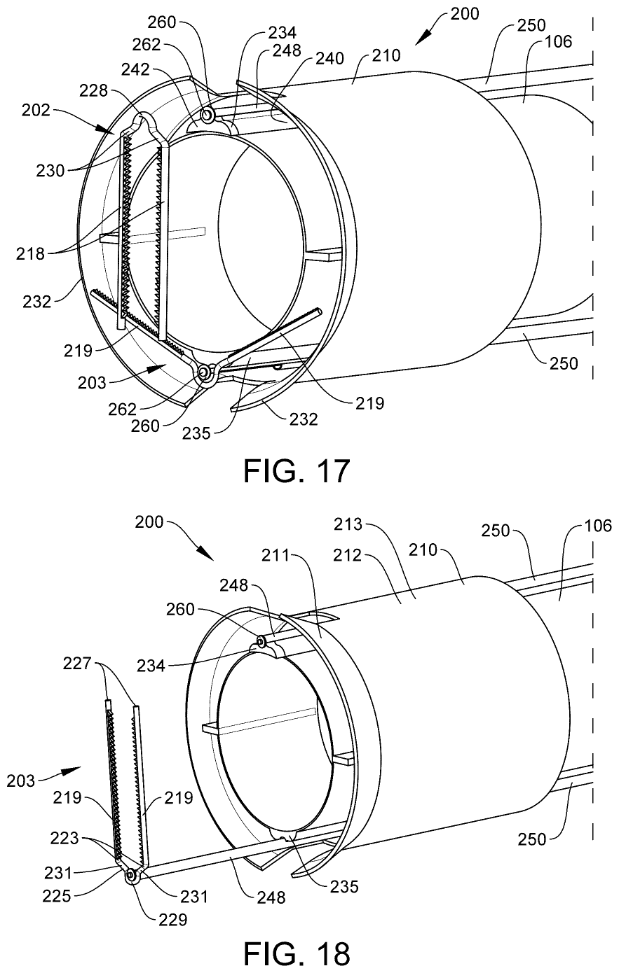

Those skilled in the art will recognize that these components do not need to be transparent to function as described herein. This is generally preferred as it can enhance the user's ability to visualize the area surrounding the distal end of the endoscope 106, but is not necessary. According to an exemplary embodiment, the first clip 202 includes clip arms 218 (e.g., two clip arms), each of which extends from a first end 222 coupled to a connection part 224 to a free second end 226. Similarly, the second clip 203 includes clip arms 219 (e.g., two clip arms), each of which extends from a first end 223 coupled to a connection part 225 to a free second end 227. The connection part 224 includes a central curved section 228 that is biased to pull the clip arms 218 toward one another so that, when not constrained by the cap 210, the clip arms 218 move to the closed tissue clipping position as shown in FIG. 17.

The connection part 224 also includes a pair of opening sections 230 configured so that, when the first clip 202 is drawn proximally into contact with the distal end 232 of the cap 210 as will be described below in more detail, contact between the opening sections 230 and the distal end 232 of the cap 210 force the clip arms 218 apart into a reload configuration in which the arms are spread apart from one another into a generally V-shaped configuration, as shown in FIG. 14. The first clip 202 of this embodiment is formed of Nitinol with a memorized shape corresponding to the tissue clipping configuration shown in FIG. 17. However, those skilled in the art will understand that any of the clips of the disclosed embodiments may be made of any of a number of suitable biocompatible materials such as, for example, stainless steel or plastics such as, for example, PEEK, Polypropylene, PU, PVC or PC.

Similarly, the connection part 225 includes a central curved section 229 that is biased to pull the clip arms 219 toward one another so that, when not constrained by the cap 210, the clip arms 219 move to the closed tissue clipping position as shown in FIG. 18. The connection part 225 also includes a pair of opening sections 231 configured so that, when the second clip 203 is drawn proximally into contact with the distal end 232 of the cap 210 as will be described below in more detail, contact between the opening sections 231 and the distal end 232 of the cap 210 force the clip arms 219 apart into a reload configuration in which the arms are spread apart from one another into a generally V-shaped configuration similar to that shown for the first clip 202 in FIG. 14. The second clip 203 of this embodiment may also be formed of Nitinol with a memorized shape corresponding to the tissue clipping configuration shown in FIG. 18.

As will be described in more detail below, the distal end 232 of the cap 210 includes features that permit a user to reload the first clip 202 or the second clip 203 onto the cap 210 if, for example, a user determines that the clip has not been placed as desired on the tissue to be clipped. Those skilled in the art will understand that the shapes of any of the described items may be changed in any manner that does not impede the functioning of the system 200 in placing and deploying the first and second clips 202, 203 as described below. For example, a cap 210 need not be circular but may be elliptical or any other shape configured to couple as desired to an insertion device 104 and to minimize trauma to any tissue contacted by the cap 210 as it is advanced to a target site within the body. Similarly, the clip arms 218 of the first clip 202 or the clip arms 219 of the second clip 203 need not be parallel or even linear when in the closed tissue clipping configuration. Rather, the clip arms 218, 219 may have spatial relation to one another (and need not be symmetric or the same size and/or shape) selected to draw tissue together as desired.

As seen in FIGS. 11-19, the outer body 212 of this embodiment includes a conically flared distal portion 211 that extends distally from a generally cylindrical portion 213. The distal portion 211 of the outer body 212 increases in diameter from a proximal end at which the distal portion 211 joins the generally cylindrical portion 213 to a maximum diameter at a distal end thereof. As would be understood by those skilled in the art, this facilitates the reloading of the first and second clips 202, 203 onto the inner body 214 because the taper of the inner surface of the distal portion 211 permits the first and second clips 202, 203 to be gradually contracted from the V-shaped reload configuration as the first and second clips 202, 203 are pulled proximally into the gap 216. It will be further understood that a similar conical distal portion may be included on the outer body 112 of the cap 110 described above and that a cap 210 including an outer body 212 without such a flared distal portion may be included in the system 200.

In this embodiment, the first and second clips 202, 203 are, in the open tissue receiving configuration, substantially circular as they are held in this shape by contact with the inner body 214 of the cap 210. This permits an interior space of the first and second clips 202, 203 to be made as large as possible without increasing a maximum cross-sectional extent of the system 200 beyond that of the circular distal end of the outer body 212 of the cap 210. Thus, in this embodiment, an outer diameter of the first and second clips 202, 203 is substantially the same as the inner diameter of the outer body 212.

The distal end 232 of the cap 210 includes a first mounting structure 234 and a second mounting structure 235 that project, in this embodiment, distally from diametrically opposed locations on the outer surface of the inner body 214. The first and second mounting structures are shaped and sized to correspond to the size and shape of the connection parts 224, 225 when the first and second clips 202, 203 are in the open, tissue receiving configuration. Thus, each of the first and second mounting structures 234, 235 includes a central section 236 having a channel configured to slidably receive the inner coil 248 and a pair of shoulders 238 projecting laterally outward from the central section 236. The shoulders 238 project radially outward from the outer surface of the inner body 214 of the cap 210 by a first distance and the central section 236 extends radially outward from the shoulders 238 by a second distance. That is, portions of the central section 236 that form the channel configured to receive the inner coil 248 extend radially outward from the adjacent portions of the central section 236.

The first and second mounting structures 234, 235, the shoulders 238 and the central sections 236 each extend from a proximal end 240 to a distal end 242 that extends distally beyond the distal end of the inner and outer bodies 214, 212, respectively, of the cap 210. In this embodiment, the first and second mounting structures 234, 235 extend to the proximal end of the cap 210. However, those skilled in the art will understand that the first and second mounting structures 234, 235 may be shortened and are required to extend along only that portion of the length of the cap 210 on which the first and second clips 202, 203 will be received during use. That is, the first and second mounting structures 234, 235 do not need to extend proximally beyond a proximal-most position of the corresponding one of the first and second clips 202, 203.

As will be discussed in more detail below, if after the corresponding one of the first and second clips 202, 203 has been advanced off of the cap 210 and clipped over tissue, a user determines that the clip is not positioned as desired, the user can draw the corresponding one of the first and second clips 202, 203 proximally until the clip contacts the corresponding one of the first and second mounting structures 234, 235. That is, because the first and second mounting structures 234, 235 project distally beyond the distal ends of the inner and outer bodies 214, 212, respectively, the first and second clips 202, 203 will first contact the corresponding first and second mounting structures 234, 235 as it is drawn proximally back toward the cap 210.

The shape of the first and second mounting structures 234, 235 are selected so that, as the connection parts 224, 225 are drawn proximally against the distal end of the corresponding first and second mounting structures 234, 235 one of the first and second clips 202, 203 is forced into an open, reloading configuration with the shoulders 238 forcing the clip arms 218, 219 apart from one another into a substantially V-shaped reloading configuration as shown in FIG. 14. As the connection part 224 is drawn further proximally over the corresponding first and second mounting structures 234, 235, the free second ends 226, 227 of the clip arms 218, 219 contact the distal end of the outer body 212 forcing the clip arms 218, 219 to bend so that the free second ends 226, 227 enter the gap 216. As one of the first and second clips 202, 203 is drawn further proximally, the clip arms 218, 219 are pulled into the gap 216 until one of the first and second clips 202, 203 returns to the open tissue receiving configuration (i.e., an insertion configuration in which one of the first and second clips 202, 203 was initially positioned on the cap 210).

The system 200 includes a mechanism for deploying the first and second clips 202, 203 that includes control wires 244 that extend from a handle similar to the handle 146 (except with separate actuators for each of the control wires 244) that remains outside the patient's body accessible to a user when the distal end of the system 200 is inserted to a target site within the body (e.g., via natural body lumen accessed via a natural body orifice). Each of the control wires 244 extends within a corresponding inner coil 248 that is slidably received within a corresponding outer coil 250. Each inner coil 248 extends from a proximal end coupled to a spool similar to the spool 152 (or any other suitable actuator) that is slidably mounted on a body of the handle.

Each inner coil 248 extends to a distal end 256 slidably received within the channel of the central section 236 of the corresponding one of the first and second mounting structures 234, 235. Each outer coil 250 is coupled to the body of the handle and extends to a distal end coupled to the proximal end of corresponding one of the first and second mounting structures 234, 235. Each outer coil 250 is configured to extend along an outer surface of the insertion device 104 with the corresponding control wire 144 extending through the corresponding inner coil 248 to couple to the corresponding one of the first and second clips 202, 203. Specifically, each of the first and second clips 202, 203 includes a wire mounting structure 260 and each of the control wires 244 includes at its distal end an increased diameter flange 262.

Each of the control wires 244 extends through the wire mounting structure 260 through a hole 264 therein so that the flange 262 is on the distal side of the wire mounting structure 260. The diameter of the flange 262 is selected to be larger than the diameter of the hole 264 so that movement of the control wire 244 proximally relative to the cap 210 pulls the first clip 202 or the second clip 203 proximally as the flange 262 pushes against the wire mounting structure 260. Each of the wire mounting structures 260 is configured so that when the corresponding control wire 244 imparts to the wire mounting structure 260 a force of no more than a predetermined level, the wire mounting structure 260 prevents the flange 262 from moving proximally therepast. However, the wire mounting structure 260 is constructed so that, when subjected by the flange 262 to a proximally directed force of greater than the predetermined level, the flange 262 will be pulled proximally through the wire mounting structure 260 to completely separate the first clip 202 or the second clip 203 from the rest of the system 200 as will be described in more detail below.

As would be understood by those skilled in the art, the predetermined force is designed to be greater than the level of force that will need to be applied to the first and second clips 202, 203 to draw the first and second clips 202, 203 proximally onto the cap 210 after the corresponding first clip 202 or the corresponding second clip 203 has been provisionally deployed (e.g., if the user wishes to remove and reposition the first clip 202 or the second clip 203). Each of the control wires 244 extends through the corresponding inner coil 248 to couple to a push button or other actuator (e.g., slidably mounted on a spool of the handle as described above in regard to the system 100).

As indicated above, each of the control wires 244 extends through a corresponding on of the inner coils 248 from a proximal end coupled to a push button or other actuator to a distal end coupled to a corresponding flange 162. The inner coils 248 pass through the channel extending along the first and second mounting structures 234, 235 and through the wire mounting structure 260 to the flange 262 which is located on the distal side of the wire mounting structure 260.

The user may open and close the first and second clips 202, 203 as often as desired by moving the corresponding inner coil 248 distally relative to the cap 210 to push the first clip 202 or the second clip 203 distally off of the cap 210 or by moving the corresponding inner coil 248 proximally relative to the cap 210 to pull the first clip 202 or the second clip 203 proximally back onto the cap 210 as described above. When the first clip 202 or the second clip 203 has been moved off of the cap 210 and has closed over the target tissue, the user may then extend the corresponding inner coil 248 distally by moving the spool distally relative to the body of the handle while simultaneously drawing the endoscope 106 and the cap 210 proximally.

This moves the endoscope 106 proximally, away from the first clip 202 or the second clip 203 which remains clipped to the tissue, to a review configuration. This, in turn, widens the field of view available to the user who may then move the endoscope 106 as desired to examine the position of the first clip 202 or the second clip 203 to determine if the clip has been deployed as desired. If the user determines that the clip has not been deployed as desired, the user may then move the endoscope 106 distally while proximally withdrawing the corresponding inner coil 248 and the corresponding control wire 244 proximally until the first clip 202 or the second clip 203 contacts the corresponding first mounting structure 234 or the corresponding second mounting structure 235.

As described above, further movement of the control wire 244 proximally at this point pulls the first clip 202 or the second clip 203 over the corresponding first mounting structure 234 or the corresponding second mounting structure 235 to spread the clip arms 218, 219 apart into the reloading configuration. Further proximal movement of the control wire 244 then pulls the clip arms 218, 219 into the gap 216 while the connection part 224 moves proximally over the corresponding first and second mounting structures 234, 235 so that the clip returns to the insertion configuration.

As indicated above, the flange 262 at the distal end of each of the control wires 244 prevents the control wire 244 from being pulled proximally through the wire mounting structure 260. After a user has positioned a first clip 202, a second clip 203 as desired (e.g., after visually confirming that a target portion of tissue has been clipped by the first and second clips 202, 203 as desired), the user may finally deploy the first and second clips 202, 203 by separating the clip from the rest of the system 200. To do this, the user holds the distal end of the corresponding inner coil 248 against the proximal surface of the wire mounting structure 260 to prevent the first and second clips 202, 203 from moving further proximally.

At the same time, the user applies proximal force to the corresponding control wire 244 by sliding the push button proximally relative to the spool until a predetermined tension is applied to the control wire 244 and the flange 262 is pulled through the wire mounting structure 260. When the flange 262 has pulled free from the first and second clips 202, 203; the first clip 202 or the second clip 203 is completely separated from the rest of the system 100 and remains closed, clipped over the target tissue under the force of its own natural bias. Those skilled in the art will understand that, after a first one of the first and second clips 202, 203 has been finally deployed over a first portion of target tissue, the user may reposition the endoscope 106 and the cap 210 adjacent to a second portion of tissue to be clipped. Due to the low profile nature of the first and second clips 202, 203, the second clip 203 may be deployed close to the first clip 202 (e.g., the aid in sealing a tissue opening only partially addressed by the first clip) or may be applied to a second portion of tissue separated from the first portion of tissue (e.g., to seal a second tissue opening) and that this may be done in the same manner described above for the first clip.

It will be apparent to those skilled in the art that various modifications may be made in

the present disclosure, without departing from the scope of the disclosure. Furthermore, those skilled in the art will understand that the features of any of the various embodiments may be combined in any manner that is not inconsistent with the description and/or the functionality of the embodiments.

1-15. (canceled)

16. A clipping device for treating tissue within a living body, comprising:
a cap configured to be coupled to a distal end of an insertion instrument, the cap including an inner body defining an inner channel and an outer body surrounding the inner body and separated from the inner body by a gap;
a first clip configured to be slidably mounted on a first mounting structure, the first clip being biased toward a closed configuration in which arms of the first clip are drawn toward one another to clip tissue received therebetween, the first clip being, in an insertion configuration, slidably received on the inner body with the arms of the first clip being held separated from one another by the inner body so that tissue drawn into the channel of the inner body is drawn between the arms of the first clip;
a first control wire extending from a proximal end configured to remain outside the living body while the insertion instrument and the cap are inserted into the living body, the first control wire extending to a distal end separably coupled to the first clip;
a first sheath slidably receiving the first control wire therein and extending to a distal end movable between an insertion position in which the distal end of the first sheath is located on the inner body and an extended position in which the distal end of the first sheath extends distally beyond the distal end of the inner body; and
a second sheath slidably receiving the first sheath and extending from a proximal end configured to remain outside the living body to a distal end fixed relative to the inner body, the first mounting structure extending distally beyond a distal end of the inner body to engage a central portion of the first clip as the first clip is drawn proximally toward the cap so that the first mounting structure forces the arms of the first clip apart from one another into a reload configuration in which the first clip may be drawn into the gap between the inner and outer bodies.

17. The device of claim 16, wherein a distal end of the outer body is configured to engage the arms of the first clip in the reload configuration to force the arms of the first clip back into the insertion configuration as the first clip is drawn proximally into the gap.

18. The device of claim 17, wherein the distal end of the outer body flares outward compared to a proximal part of the outer body.

19. The device of claim 16, wherein the arms of the first clip are substantially linear in the reload configuration and wherein the first mounting structure is configured to engage the central portion of the first clip as the first clip is drawn proximally over the first mounting structure so that the arms of the first clip are separated from one another into a V shape in the reload configuration.

20. The device of claim 16, wherein the channel of the inner body is configured so that, when the cap is mounted on an insertion device as desired, the distal end of the insertion device is open to the channel and wherein the channel extends to an open distal end so that tissue drawn into the channel is drawn between the arms of the first clip when the first clip is mounted on the inner body in the insertion configuration.

21. The device of claim 16, wherein the inner and outer bodies are formed of a transparent material.

22. The device of claim 16, wherein the central portion of the first clip is coupled to a first one of the arms of the first clip via a first connection part and the central portion of the first clip is coupled to a second one of the arms of the first clip via a second connection part, the central portion of the first clip being configured to bias the first clip toward the closed configuration and the first and second connection parts of the first clip being configured to engage laterally outer portions of the first mounting structure to drive the first clip to the reload configuration.

23. The device of claim 16, wherein the first clip includes a wire mounting structure having an opening through which the first control wire passes to separably couple the first clip to the first control wire, the first control wire including a flange at a distal end thereof that is larger than the opening in the wire mounting structure of the first clip.

24. The device of claim 23, wherein the wire mounting structure is designed to fail when subject to a proximally directed force of at least a predetermined level by the flange of the first control wire.

25. The device of claim 24, wherein the predetermined level of the proximally directed force is less than an amount of a proximally directed force required to draw the first clip proximally onto the inner body.

26. The device of claim 24, wherein the first sheath is extendable into contact with a proximal surface of the wire mounting structure of the first clip to prevent proximal movement of the first clip so that a proximally directed force applied to the first control wire can be raised above the predetermined level.

27. The device of claim 16, wherein the inner body further includes a second mounting structure, the device further comprising:
a second clip configured to be slidably mounted on the second mounting structure, the second clip being biased toward the closed configuration in which arms of the second clip are drawn toward one another to clip tissue received therebetween, the second clip being, in an insertion configuration, slidably received on the inner body with the arms of the second clip being held separated from one another by the inner body so that tissue drawn into the channel of the inner body is drawn between the arms of the second clip;
a second control wire extending from a proximal end configured to remain outside the living body while the insertion instrument and the cap are inserted into the living body, the second control wire extending to a distal end separably coupled to the second clip;
a third sheath slidably receiving the second control wire therein and extending to a distal end movable between an insertion position in which the distal end of the third sheath is located on the inner body and an extended position in which the distal end of the third sheath extends distally beyond the distal end of the inner body; and
a fourth sheath slidably receiving the third sheath and extending from a proximal end configured to remain outside the living body to a distal end fixed relative to the inner body, the second mounting structure extending distally beyond a distal end of the inner body to engage the central portion of the second clip as the second clip is drawn proximally toward the cap so that the second mounting structure forces the arms of the second clip apart from one another into a reload configuration in which the second clip may be drawn into the gap between the inner and outer bodies.

28. The device of claim 27, wherein the first and second mounting structures are located on the inner body diametrically opposed to one another.

29. A clipping device for treating tissue within a living body, comprising:
a cap configured to be coupled to a distal end of an insertion instrument, the cap including an inner body defining an inner channel and an outer body surrounding the inner body and separated from the inner body by a gap, the inner body further including a first mounting structure extending over a first portion of a circumference of the inner body and projecting distally beyond a distal end of a second portion of the circumference of the inner body;
a first clip configured to be slidably mounted on the first mounting structure, the first clip being biased toward a closed configuration in which arms of the first clip are drawn toward one another to clip tissue received therebetween, the first clip being, in an insertion configuration, slidably received on the inner body with the arms of the first clip being held separated from one another by the inner body so that tissue drawn into the channel of the inner body is drawn between the arms of the first clip, the first mounting structure being configured to engage a central portion of the first clip as the first clip is drawn proximally toward the cap so that the first mounting structure forces the arms of the first clip apart from one another into a reload configuration in which the first clip may be drawn into the gap between the inner and outer bodies; and
a first control wire extending to a distal end separably coupled to the first clip, the first control wire being operable to move the first clip proximally and distally relative to the inner body to move the clip between the insertion configuration, the closed configuration and the reload configuration.

30. The device of claim 29, wherein a distal end of the outer body is configured to engage the arms of the first clip in the reload configuration to force the arms of the first clip back into the insertion configuration as the first clip is drawn proximally into the gap.

31. A method for clipping tissue comprising:
inserting to a target location within a living body a clipping device, comprising:
a cap coupled to a distal end of a flexible insertion instrument with a first clip stretched over an inner body of the cap in an insertion configuration, wherein the inner body further includes a first mounting structure extending over a first portion of a circumference of the inner body and projecting distally beyond a distal end of a second portion of the circumference of the inner body;
drawing a portion of tissue into a channel defined by the inner body so that a first portion of tissue is drawn between arms of the first clip;
pushing the first clip distally so that the arms of the first clip are drawn together under a natural bias of the first clip to clip the portion of tissue drawn into the channel;
observing a position of the first clip on the portion of tissue to determine if a first target tissue has been clipped as desired and, when the first target tissue has not been clipped as desired, drawing the first clip proximally so that engagement between a central portion of the first clip and the first mounting structure forces the arms of the first clip apart from one another into a reload configuration to release the portion of tissue;
drawing the first clip further proximally into a gap extending between the inner body and an outer body of the cap, engagement between the arms of the first clip and the outer body forcing the first clip back into the insertion configuration;
repositioning the cap relative to the first target tissue and drawing into the channel a further portion of tissue; and
pushing the first clip distally so that the arms of the first clip are drawn together to clip the further portion of tissue.

32. The method of claim 31, wherein the clipping device includes a first control wire connected to the first clip for moving the first clip between the insertion configuration, a closed configuration and the reload configuration further comprising, when it is desired to fully deploy the first clip, applying to a connection between the first control wire and the first clip a tension greater than a first predetermined tension to permanently separate the first clip from the first control wire.

33. The method of claim 32, wherein the first predetermined tension is greater than a tension applied to the connection between the first control wire and the first clip when the first clip is drawn proximally from the closed configuration to the reload configuration and a tension applied to the connection between the first control wire and the first clip when the first clip is drawn proximally from the reload configuration to the insertion configuration.

34. The method of claim 31, wherein the wherein the inner body further includes a second mounting structure and wherein the clipping device further includes a second clip mounted on the second mounting structure in an insertion configuration with the arms of the second clip held separated from one another by the inner body, the method further comprising:
drawing into the channel a still further portion of tissue;
operating a second control wire coupled to the second clip to push the second clip distally off of the inner body so that the arms of the second clip close over the still further portion of tissue;
observing a position of the second clip on the still further portion of tissue to determine if a second target tissue has been clipped as desired and, when the second target tissue has not been clipped as desired, operating the second control wire to draw the second clip proximally so that engagement between a central portion of the second clip and the second mounting structure forces the arms of the second clip apart from one another into a reload configuration to release the still further portion of tissue;
operating the second control wire to draw the second clip further proximally into the gap between the inner body and the outer body of the cap, engagement between the arms of the second clip and the outer body forcing the second clip back into the insertion configuration;
repositioning the cap relative to the second target tissue and drawing into the channel an additional portion of tissue; and
operating the second control wire to push the second clip distally so that the arms of the second clip are drawn together to clip the additional portion of tissue.

35. The method of claim 34, further comprising operating the second control wire to apply to a connection between the second control wire and the second clip a tension greater than a predetermined tension to separate the second clip from the second control wire.
